(19) United States
(12) Patent Application Publication
(10) Pub. No.: US 2006/0216264 A1
(43) Pub. Date: Sep. 28, 2006

(54) Eurpoean slenderizing seaweed bath

(76) Inventor: Amalia Donham Maguire, Goleta, CA (US)

Correspondence Address: AMALIA DONHAM MAGUIRE, 574 POPPYFIELD PLACE, GOLETA, CA, 93117, US

(21) Appl. No.: 11/367,147

(22) Filed: Feb. 13, 2006

Related U.S. Application Data
(60) Provisional application No. 60/594,281, filed on Mar. 24, 2005.

(51) Int. Cl. A61K 8/97; A61K 36/63

(52) U.S. Cl. 424/74; 424/769

(57) ABSTRACT

Italian Secret Olive Butter Body Crème uses modern science and old world Italian traditions to create a unique skin moisturizing product that provides a lasting effect. The combination of naturally occurring anti-oxidant and moisturizing elements produces a healthful benefit to the skin. Ingredients include: Natural Olive Butter, Shea Butter, Organic Green Tea, Sweet Almond Oil, Vitamin E, Vitamin C, and Essential Oils in a single, easy-to-use, daily skin moisturizer.

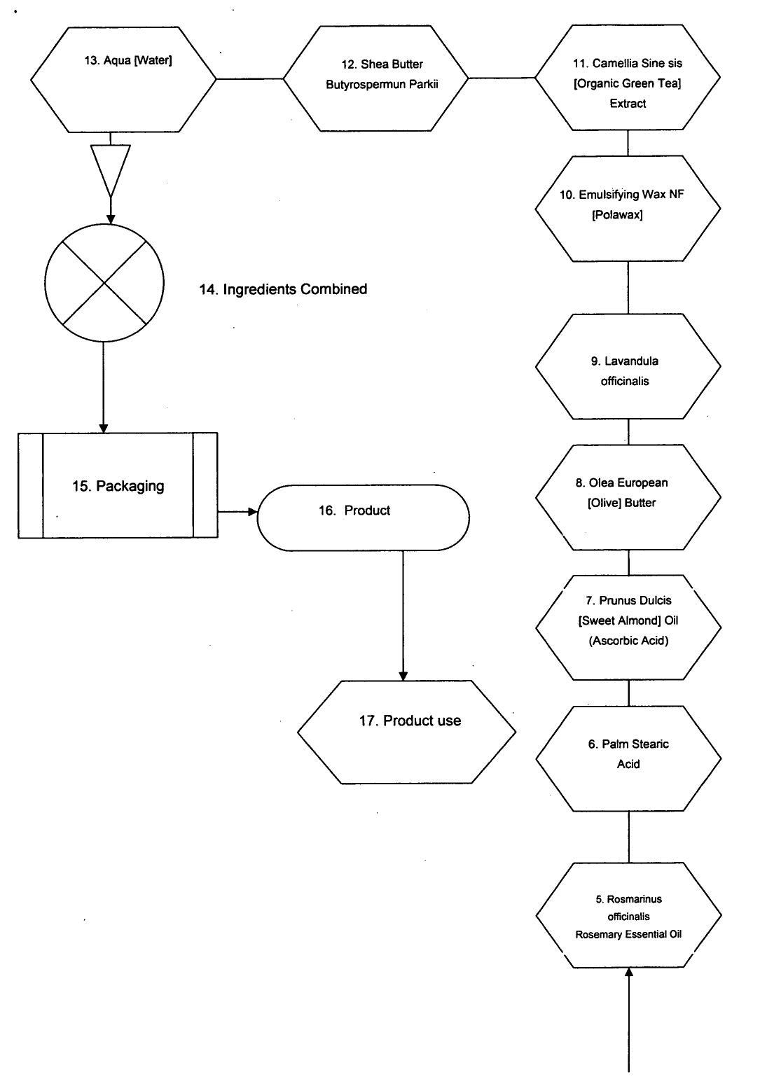

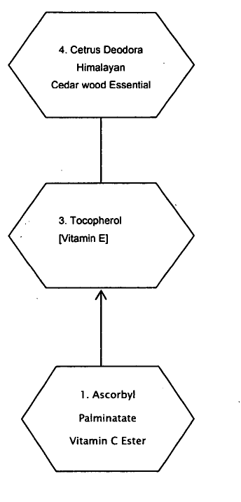

### DESCRIPTION

This invention relates to Italian secret olive butter body crème. It contains the following components: Water, Shea Butter, Organic Green Tea, Emulsifying Wax NF, Lavender Essential Oil, Olive Butter, Sweet Almond Oil, Palm Stearic Acid, Rosemary Essential Oil, Himalayan Cedar wood Essential Oil, Vitamin E Oil, Vitamin C.

Shea Butter (also known as Karite), has healing properties in its natural form (included in this product) is made up of Vitamins A, E, and Cinnamic acid. Shea butter has been used for hundreds of years on excessively dry skin. Shea Butter helps to moisturize the skin and reduce daily inflammation, increase the speed of healing of wounds. Used to heal severely dried skin. Shea Butter contains beneficial vegetable fats that promote cell regeneration and circulation. Shea Butter also contains natural UV protection.

Olive Butter is for moisture and Antioxidant effects. Olive Butter supplies squalene, a naturally occurring substance found in many plants. Squalene is the principle liquid and emollient found in human skin. Squalene keeps skin soft, healthy, and at a natural balance with antioxidant capabilities to protect from environmental factors such as sun, pollution, and chemicals.

Sweet Almond Oil is obtained from died kernels of the almond tree. Almond Oil is high in minerals, protein and naturally-occurring vitamins, including A, B1, B2, B6, and E. Sweet Almond Oil is added for these properties, but also its high rate of absorption.

Vitamin E is an anti-oxidant. Sweet Almond oil is an unsurpassed nutrient for softening and conditioning skin. Green Tea is known for anti-oxidant effects. Vitamin C has anti-aging and anti-oxidant properties as well. Essential oils are added for appealing odor and aromatherapy benefits.

Vitamin C oral supplementations are helpful, although our topical preparation is able to deliver high dosages and more realized benefit to the skin area it is applied. Studies have shown that Vitamin C stimulates collagen production and may minimize fine lines and sun skin damage.

These ingredients work perfectly together because all have similar moisturizing and anti-oxidant properties. The key is the combination of all of the ingredients together in the concentrations indicated. Many of these ingredients, including Shea Butter contain Vitamin E, in various compounds, including alpha, beta, delta, and gamma Tocopherol. The Vitamin E family is known for its antioxidant capabilities. Anti-oxidants neutralize free radicals, which are unstable molecules produced from a variety of unhealthy sources including air pollution. Topical Vitamin E has been shown in numerous studies to reduce the effects of psoriasis and sun damage to the skin. Our Vitamin E is completely absorbed immediately and to its best effect.

### PURPOSE/GOAL OF INVENTION

The purpose of the invention is to provide a single skin moisturizing and antioxidant product, encompassing a unique set of ingredients, for those that suffer from very dry skin. In addition, presenting this solution with elements in their natural state is a priority.

### PRODUCT SPECIFICATIONS

Olive Butter Body Crème is all natural pure skin moisture is an ultra-rich formulation with the consistency of whipped cream that sinks luxuriously into the skin to help quickly relieve your dry, parched skin. This Body Butter Crème works on the entire body. This product utilizes several natural ingredients known to produce desired skin treatment results.

### BRIEF DESCRIPTION: DRAWINGS

The drawing on the following page depicts:

A. The gathering of the 13 essential ingredients

B. Combining the ingredients to produce the desired effect

C. Mixture is packaged with pump dispensing device.

D. End user acquires product

E. End user applies crème product to the skin as desired.

What is claimed:

1 Italian Secret Olive Butter Body Crème will provide unsurpassed moisturizing and emollient results, due to its unique combination of antioxidant and natural moisturizing ingredients. The user applies the product after shower or bath to retain and promote skin moisturizing. Best results are achieved and full benefits of the product realized when the user exfoliates the skin before the application of the Olive Butter Body Crème. Continued use may lead to more moisturized and healthy looking skin.
